(19) United States
(12) Patent Application Publication
(10) Pub. No.: US 2005/0027187 A1
(43) Pub. Date: Feb. 3, 2005

(54) Process for the coupled display of intra-operative and interactively and iteratively re-registered pre-operative images in medical imaging

(76) Inventors: Karl Barth, Hochstadt (DE); Gerd Wessels, Effeltrich (DE)

Correspondence Address: SCHIFF HARDIN LLP;Patent Department, 6600 Sears Tower, 233 South Wacker Drive, Chicago, IL, 60606, US

(21) Appl. No.: 10/897,673

(22) Filed: Jul. 23, 2004

(30) Foreign Application Priority Data
Jul. 23, 2003 (DE) 103 33 543.9

(51) Int. Cl. A61B 5/05

(52) U.S. Cl. 600/407

(57) ABSTRACT

A method couples the display of intra-operative and interactively and iteratively re-registered pre-operative images in medical imaging and includes: recording a pre-operative image of a region of a patient that is of interest, recording an intra-operative image or an image sequence of the region of interest, performing a registration of the pre-operative image with the intra-operative image, coupling a display of the pre-operative image and the intra-operative image, re-registration of the pre-operative image, in case the pre-operative image and the intra-operative image do not match, and repeating steps of coupling the display and re-registration until an acceptable match is present.

### BACKGROUND OF THE INVENTION

The present invention involves a process for the coupled display of a 2-D or 3-D image recorded pre-operatively with any desired imaging modality using an intra-operative 2-D or 3-D image, obtained, for example, using a video camera or an ultrasound device. The invention particularly involves an interactive and possibly iterative comparing of the pre-operative image to the intra-operative image.

Examinations and treatments of a diseased patient are being done in increasing quantity in a minimally invasive manner, i.e., with as little traumatization of the patient as possible. For example, the diagnostics are performed with endoscopes or laparoscopes. Endoscopes are introduced through natural access pathways (i.e. esophagus, intestine) into the examination area of the patient and send images to the doctor on site from the access channel. The images, as in the case of ultrasound, for example, can also contain objects, e.g., organs, outside the channel.

From a surgical perspective, it is urgently desired to display, in a coupled manner, “pre-operative” 2D or 3D images obtained in the context of a preliminary examination with the “intra-operative” images recorded during an intervention. “Coupled”, in this context, means that the images are brought together in some manner in relation to each other, e.g., shown next to each other or as an overlay, etc. In the pre-operative images, this can involve images of any image modalities, for example, computer tomograms (CT-images), nuclear medical records, positron emission tomograms (PET), magnetic resonance tomograms (MRT) or 2D-3D-X-rays (C-arc images). Pre-operative images of this type can also be combinations of 2D and 3D images. Also included among them are the fusion of images of the imaging modalities also mentioned (2D-2D-fusion, 2D-3D fusion, 3D-3D fusion, for example, in the form of combined CT- and PET-images). Pre-operative images of this type, usually high-resolution, should be assigned to intra-operative images and/or directly superimposed on them.

A first explicit image is ultrasound images superimposed with multi-planar reformatting (MPR), which were obtained by computer tomography. The MPR layers are, in the process, oriented parallel to the scanning bays of the ultrasound device.

A second explicit example is endoscopic/laparoscopic images (video images), which are superimposed with 3D-volume rendering views (3D-display of a body region) related in the same viewing cone and/or on screen edges in the same rectangular viewing pyramids. The direct superimposition of video images of this type requires, however, a pre-processing to compensate for the camera distortion of these images, since the lens image of the camera usually leads to a “pin cushion distortion”.

In order to be able to create a site-accurate simultaneous display and/or a correct (locationally correct) superimposition of intra-operative images with pre-operative images, it is necessary to register the intra-operative image (which contains at least one object of interest) and the pre-operative image and/or both images with regard to each other and/or each with regard to the coordinate systems of an intra-operatively used navigation system. Registering two image data sets (three-dimensional and/or two-dimensional in nature) means determining an imaging specification which carries the coordinates of one system into the coordinates of another system. In general, an imaging specification of this type and/or registration is produced by a registration or transformation matrix.

In order to register two images, different possibilities are conceivable:

1. It is possible to identify one or more image elements in the pre-operative image and identify the same element(s) in the intra-operative image or object, and then orient one of the two—preferably the pre-operative image—by at least one of translation, rotation, projection, and scaling relative to the other object/image—preferably the intra-operative object/image. Image elements identified in this way are identified as “markers” and can be applied from anatomical origin or artificially.

Markers of anatomical origin—such as vascular bifurcation points, small sections of coronary arteries, but also angle of the mouth or tip of the nose—are identified as “anatomical markers”. Artificially introduced or mounted marking points are identified as “artificial markers”. Artificial markers are, for example, objects which are affixed (e.g., adhered) to the surface of the body, and, for the sake of precision, preferably to non-movable points, e.g., to the pelvic bone. Anatomical and artificial markers can be affixed by the user interactively in the pre-operative image (e.g., by clicking on the screen) and are re-identified in the intra-operative object/image. Instead of interactive allocation, an automatic determination is also possible in two images using suitable algorithms. A registration of this type is identified as “marker-based registration”.

2. An additional possibility is “image-based registration”, in which the pre-operative image and/or a part of it is compared for its agreement with the intra-operative image and/or a part of it, so that in order to optimize the agreement, for example, the pre-operative (partial) image is modified by translation and/or rotation and/or extension relative to the intra-operative image until the agreements of the two images reach a predefined minimum quantity.

As a dimensional quantity here, for example, the 2D- or 3D-correlation is used or, for example, the Venot-algorithm that is faster with regard to the computation time. Functionally, in the process, the inter-operatively used camera is at first brought into a user-guided position in which the intra-operative image is similar to the pre-operative image and then first initiates the optimization cycle in order to shorten the computation time for the registration. This means no special additional expense, but instead fits into the work flow, because in this way, the instrument with the camera is already “on-site”.

A site-accurate coupled display of pre-operative and/or intra-operative images in the form of simultaneous displays and/or overlays is a special concern for exact registration. For coupling images of exclusively rigid objects, the registration goal has been achieved. The problem that has not yet been solved from a medical-technical perspective in a registration procedure is presented by movable, shiftable objects, as, for example, are produced by deformable organ structures.

An example of this is “brain-shift”: though the skull of a patient can be almost perfectly registered and also fixed, the brain or parts of it move during a paracentesis (due to the loss of cerebrospinal fluid/liquor). It also occurs that during an examination and/or operation, a repositioning of the patient is necessary and, in the process, parts of the brain move, are additionally compressed, or expand.

Even more pronounced are, for example, organ shifts in the abdominal region, where mechanical effects are produced during an operation. Particularly during minimally invasive interventions, shifts and volume changes of gas-filled structures also result due to inflating of the fore-abdomen (peritoneum) with carbon dioxide for the purpose of the organs present which have relatively little supporting tissue. Here, it is not possible, based on the state of the art, to identify reliably artificial or anatomical markers and/or surface structures (automatically) inside the body with a restricted view according to a marker or image-based registration.

Organ shifts and/or organ deformations of this type between pre-operative and intra-operative images cause a distinct inequality and/or variability of both images, as a result of which a clear comparative image display, particularly a fusion of both images, is no longer possible.

### SUMMARY OF THE INVENTION

The purpose of the present invention is thus to provide a process by which a reaction can be made to the organ shifts and/or organ deformations between images obtained pre-operatively and intra-operatively, and an agreement can be created permitting a coupled display of both images that is logical from a diagnostic standpoint.

This purpose is achieved by a method for the coupled display of intra-operative and pre-operative images in medical imaging, comprising: recording a pre-operative image of a region of a patient that is of interest; recording an intra-operative image or an image sequence of the region of interest; registering the pre-operative image with the intra-operative image; coupling a display of the pre-operative image and the intra-operative image; re-registering the pre-operative image, in case the pre-operative image and the intra-operative image do not match; and repeating the steps of coupling the display and re-registering until an acceptable match of the pre-operative image and the intra-operative is present.

This purpose is also achieved by an apparatus configured to couple a display of intra-operative and pre-operative medical imaging images, comprising: a pre-operative recording device configured to record a pre-operative image of a region of a patient that is of interest; an intra-operative recording device configured to record an intra-operative image of the region of the patient; a display configured to present the pre-operative image and the intra-operative image to a user; a mechanism configured to couple the display of the pre-operative image and the intra-operative image; and an input device configured to allow the user to re-register the pre-operative image in case the pre-operative image and the intra-operative image do not match.

The process for the coupled display of intra-operative and pre-operative images in medical imaging includes:
taking a pre-operative image of an region on a person that is of interest,
taking an intra-operative image or an image sequence of the region that is of interest,
performing a registration of the pre-operative image with the intra-operative image,
a coupled display of the pre-operative and intra-operative image,
re-registration of the pre-operative image in case the pre-operative and intra-operative image do not agree, advantageously in one dimension,
repetition of the steps of coupled display and re-registration until an acceptable agreement is present, possibly in additional dimensions.

The registration can be done according to the invention in an image-based or marker-based manner.

In case of an image-based registration, the markers of the region of the patient to be examined are advantageously applied prior to recording the pre-operative image. Advantageously, the examination of the agreement of the pre-operative and the intra-operative image is done by computer and automatically using an algorithm on a computer. It is also advantageous that the step of re-registration contains a 3D-rotational correction and/or a shift-correction and/or a resealing correction.

In a possible embodiment form of the invention, the re-registration with resealing correction only relates to a region within the image, so that at the same time, the regions outside of this area remain as originally scaled and possibly as originally positioned.

In an additional embodiment form of the invention, the re-registration of the pre-operative image and the coupled display relate to several intra-operative images that were recorded during a periodic physiological movement cycle at defined points in time and/or locational points.

According to an embodiment of the invention, from the re-registered pre-operative images, a video sequence is obtained by interpolating based on the physiological signal between the re-registered images.

The fundamental cycle is thus advantageously defined as a corresponding physiological signal by an EKG, a blood pressure curve or a pneumatogram.

The coupled display is done either by the adjacent coordinated display of both images or instead by superimposing and/or overlay of both images. In an advantageous way, a pre-operative image and an intra-operative image present a 3D-image. The pre-operative image may be recorded advantageously with magnetic resonance tomography, computer tomography, ultrasound, positron emission tomography, or nuclear medicine processes.

The intra-operative image is usually recorded with an endoscope, laparoscope, ultrasound device, C-arc device, magnetic resonance tomography, computer tomography, positron emission tomography, or nuclear medicine processes.

In addition, a device is considered that is suitable to perform a process according to the process steps named above.

### DESCRIPTION OF THE DRAWINGS

Additional advantages, properties and characteristics of the present invention will now be explained in greater detail using embodiment examples with reference to the accompanying drawings.

FIG. 1 is a pictorial schematic diagram relating to the recording of a pre-operative or intra-operative image with a subsequently coupled display of both images in which the pre-operative image is re-registered;

FIG. 2 is a flowchart showing an overview of a conventional process for coupled display of pre-operative and intra-operative images in the flow diagram with a last iterative step of re-registration according to an embodiment of the invention; and

FIG. 3 is a flowchart showing the step that is implemented according to the invention in the iterative step S8 of the conventional flow of FIG. 2.

### DETAILED DESCRIPTION OF THE PREFERRED EMBODIMENTS

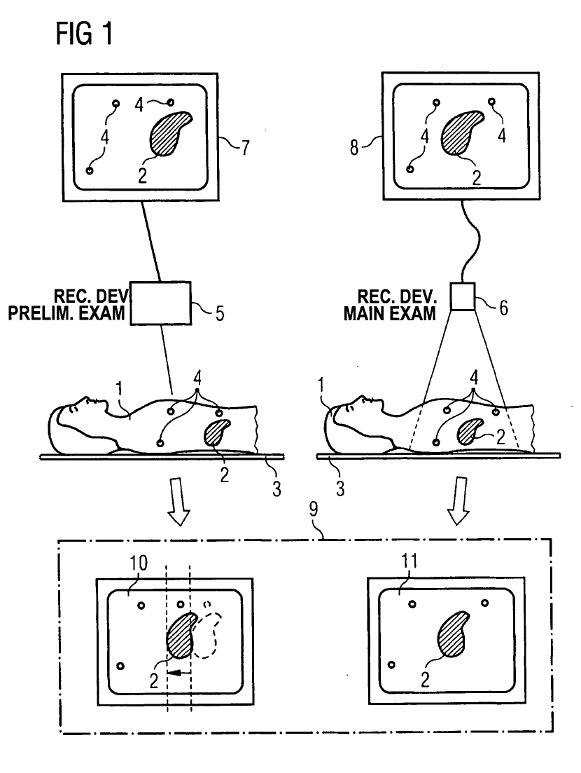

FIG. 1 shows a schematic diagram relating to the recording of pre-operative and/or intra-operative images with a subsequently coupled display 9 of both images in the lower region of the diagram. On the left side of FIG. 1, a patient 1 is shown who is located on a patient's bed 3. The region that is of interest and is to be recorded is the breast and stomach area, especially a movable schematically shown organ 2 (for example, the liver).

For the pre-operative image recording, three artificial markers 4 were used, which can be depicted with the anatomical region that is of interest—especially with the organ 2—using any desired imaging modality 5 (e.g. CT, MRT, PET, C-arc, etc.) on a monitor 7 in a 3D-image or in a selected layer of a 3D-image. In the display on the monitor 7, the organ located on the right edge of the image directly beneath the upper right marker. The display on the monitor 7 shows the pre-operative image.

The right side of FIG. 1, schematically shows how the same anatomical region is displayed intra-operatively with a recording device, for example, an ultrasound device or a laparoscope 6—i.e., at a later point in time during an intervention. The recording device was registered for this purpose at the beginning of the operation with regard to the marks present on the body and is tracked continuously in its position and orientation. Thus, the image is continuously displayed on the monitor 7 in the same position and orientation so that at least the marks on the intra-operative display 8 appear in the same position as on the display 7 with the pre-operative image. Also, an overlay of the two displays is possible on one monitor.

The organ 2 can however, e.g., as on the display 8, have been moved to the left on the direction of the patient's head, e.g., due to the shiftability of soft tissue, and thus appears in the presentation shifted on the display 8 towards the display 7.

The marker-based “base registration” is useful in spite of this, since it makes possible in advance a coarse fundamental orientation and positioning of both images (in the centimeter range).

In the most general case, an observed organ can be shifted, rotated, and compressed or expanded. In order to be able to realize a coupled display in the manner described above, the significant step of the process according to an embodiment of the invention consists in supplementing the aforementioned basis registration interactively by a so-called “post-” or “re-registration”, whereby the observed organ is changed in the pre-operative image by rotation and/or shifting and/or scaling such that the pre-operatively obtained organ image agrees with the (intra-operatively obtained) real image and if necessary, can be fused with it. The examination of the agreement of the pre-operative and intra-operative image is done either visually by the user (generally the doctor) or advantageously by computer and automatically using an algorithm on a computer.

In the lower part of FIG. 1, there is a coupled display 9 of the two images (pre-operative image 10 left, intra-operative image 11 right). The pre-operative image 10 was re-registered, i.e., the image was changed so that the position and orientation of the organ 2 of interest is identical to the display of the organ in the intra-operative image 11 on the right side. In the case of FIG. 1, the re-registration consisted of a simple shift that is symbolized by the arrow and by the dashed representation of the organ. In the most general case of the re-registration, the organ must be shifted, rotated, and scaled. Usually, in such a re-registration, the display of the organ environment is also changed accordingly as, for example, the shift of the directly adjacent marker shows.

In a re-registration, which includes, for example, a scaling due to the identification of two new positions (e.g. upper and lower edge of the liver), the re-scaling can be limited in a simple manner only to the area between the positions (e.g., the liver).

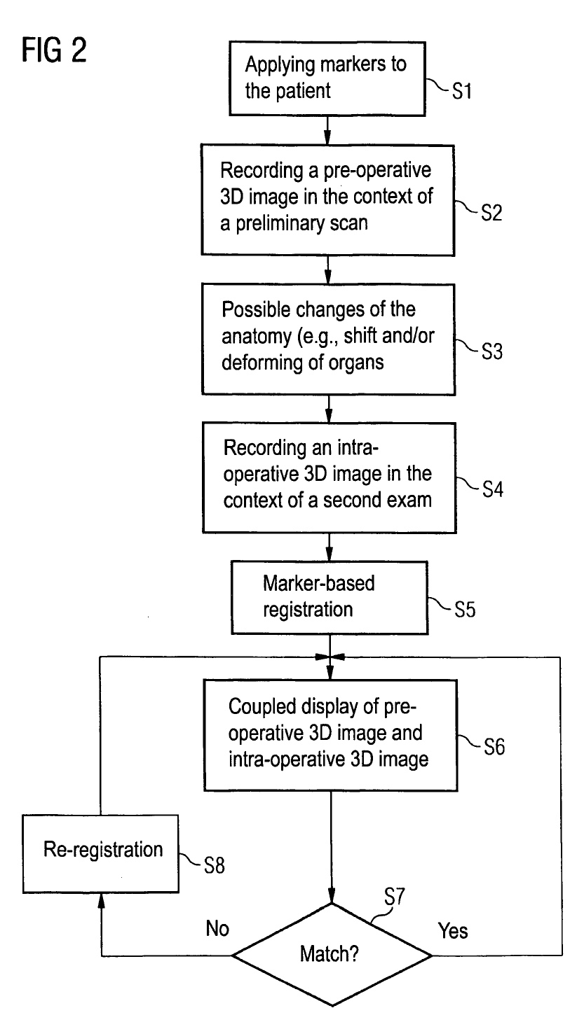

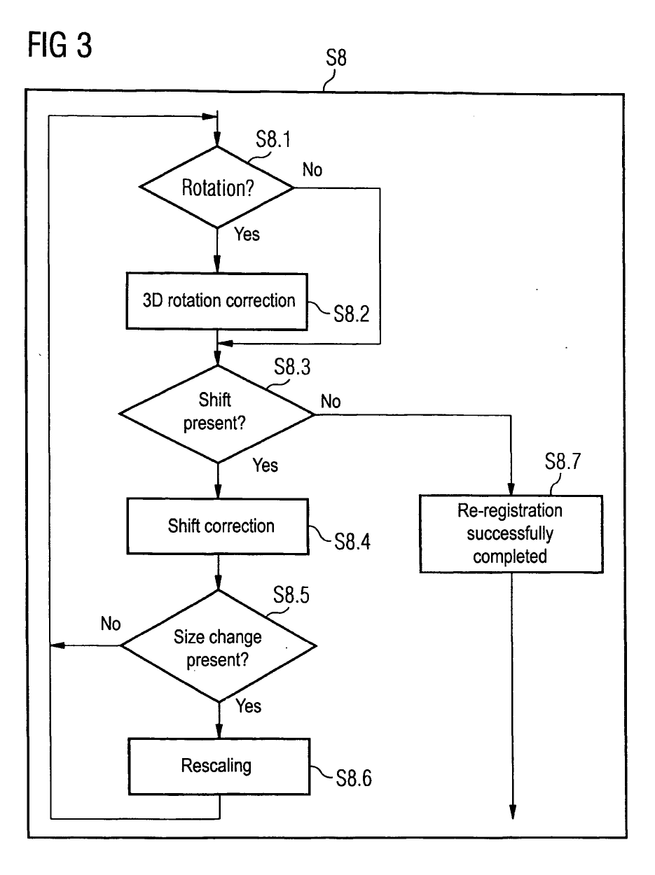

The exact sequence of the process according to the invention, especially the step of the re-registration, is shown using the process flow diagrams in FIGS. 2 and 3:

According to FIG. 2, in a first pre-operative step S1 of the process according to an embodiment of the invention, in the area of interest of the patient to be examined and/or treated, for example, artificial markers are applied and/or anatomical markers are identified. The movable organ of interest (there may also be more than one) is located in the marked area, usually a cuboid-shaped volume of, for example, approx. 50×50×50 cm.

In a second step S2, an image is recorded pre-operatively—i.e., in the context of a pre-examination—using any imaging modality, preferably a 3D-image of the marked region. After recording the pre-operative image, (in a process described as step S3), a shift, rotation, and/or deformation of the aforementioned organ occasionally occurs. Examples for this were previously mentioned (brain, liver, etc.). In a second examination S4, for the most part the main examination and/or operation, which occurs under certain circumstances several days after the pre-examination S2, an image of this same marked body region is recorded intra-operatively—i.e., in the context of an intervention. Intra-operative imaging modalities are usually endoscope, laparoscope, and/or ultrasound device, possibly also an X-ray C-arc, a CT or MR scanner, etc.

In order to orient the pre-operative image from step S2 according to the intra-operative image from step S4, a marker-based registration of both images occurs in a fifth step S5, for example. In an additional step S6, pre-operative and intra-operative images are shown coupled using a navigation system, for example, overlain, next to each other, on top of each other, etc. in the same monitor window.

In the next step S7, a check is made, based on the coupled display, whether and to what extent there is an agreement of the pre-operative image with the intra-operative image. The examination is done by the user, generally by the doctor. For complete agreement and/or agreement accepted in a possible tolerance range for both images, either a new intra-operative image can be recorded (automatic continuation, e.g., of a video sequence in the “coupled” display) and the process continued in the loop or instead the process can be modified. In case of divergence of both images because of a position and/or location change of the organ to be examined, the user can perform a “re-registration” in an additional step S8. For this purpose, the pre-operative image is changed by the user interactively (for example, via an interface with the mouse) and iteratively until, in step S7, a tolerable agreement with the intra-operative image is achieved.

Step S8 of the re-registration is shown in greater detail in FIG. 3. At first, for example, in a step S8.1, a check is made as to whether the observed organ has rotated in the time period between pre-operative and intra-operative image recording. If there is a rotation, then, in a step S8.2, in the pre-operative image, a 3D rotation correction is performed. In principle, a rotation correction S8.2 of this type can be divided up into three rotational degrees of freedom. It is simpler to perform the complete rotation correction with only one handling. This is done, for example, with a special input device such as a roller bearing or with a conventional computer mouse. In the case of a computer mouse, the rotation is currently occurring around the instantaneously vertical screen axis, coupled to a mouse movement in the x-direction (horizontal), and around the screen rotational axis, coupled to a mouse movement in the y-direction (vertical).

If the step of the 3D rotational correction S8.2 has ended, whereby the pre-operative image and/or the organ in the pre-operative image has rotated so that in the coupled display in both images, the same spatial orientation of the organ has been achieved, then, in an additional step S8.3, a check is made as to whether the organ of the pre-operative image has shifted relative to the intra-operative image. If a shift is present, then it is corrected in a next step S8.4. If, in addition, there is also a size change between the organ shown in both images—which is checked in an additional step S8.5—then, in step S8.6, an adaptation of the size ratios is performed which also includes a resealing.

The steps of the shift and resealing can occur in the three spatial directions x, y, and z. In severe cases, the steps S8.1 and S8.6 can be repeated as long as necessary until no more corrections are necessary and in step S8.7, the re-registration can be considered to have successfully ended. In this case, the pre-operative image is modified by the process step S8 to such an extent that in a coupled display of both images, a congruence of the corresponding organ is present and thus, in particular, an overlay of both images leads to a diagnostically logical result.

The coupled display can additionally be dynamically designed whereby the re-registration is done for several time points and location points of a periodic shift and it can be analytically (e.g., linearly) interpolated in between. The specification of the intra-operative images functioning as “support points” is done coupled to another signal that characterizes the movement, such as, for example, an EKG, a blood pressure curve, or a pneumatogram. This signal controls the reproduction of the pre-operative images “dynamically registered” and supplemented by interpolation as a video sequence.

The following embodiment examples explain the process according to the invention:

When using an endoscope or laparoscope, the recording of the intra-operative image is done by a video camera with an objective front lens on the instrument tip. The objective focal length can possibly be changed continuously changed via an operating element.

In the case of a coupled MPR-display, the marker-based registration and the steps of re-registration are followed by an adjustment of the intra-operatively displayed MPR layer depth (from the tip of the instrument) in the pre-operative image—including the magnification that fits the objective focal length. For this purpose, an automatic or manual adjustment option is also possibly provided for the pre-operative image, e.g., a sliding controller. The sliding controller is moved accordingly as far until, for example, an optimal correlation/congruence is created visually between the pre-operative and intra-operative image. The overlay is done in color in the (intra-operative) video image, for example, and in black-and-white in the pre-operative MPR-image (CT, MRT, PET, C-arc). By a mixing level of, for example, 50% each, both portions of the image can be differentiated very well. As an alternative, an image-based registration is also possible (determining the optimal correlation) between the intra-operative image (video image) and the pre-operative image (optimal MPR layer).

To support minimally invasive interventions in surgery and in gastro-enterology, an ultrasound device is also being used more and more often, transcutaneously and/or laparoscopically and/or endoscopically. The patient is at first registered based on at least three markers as if no shift had occurred since the preliminary examination—for example, the liver because of respiration. By the registration, the transformation matrix is obtained, whereby in the following, the navigation (in the presence of a navigation system) is coupled to the ultrasound head that is used to form an image.

In case the position of the navigation sensor is not identical to the ultrasound head and/or instrument tip, the so-called tip-transformation is to be taken into consideration. By the navigation control of the ultrasound head, for each intra-operative ultrasound image, a corresponding section is obtained through the previously recorded pre-operative (3D) image that had been acquired with corresponding high-resolution imaging modality (e.g. CT, MRT, etc.). This corresponding section is, for example, any diagonal section in the form of a so-called free multi-planar reformatting (MPR) or a perspective 3D-display according to a Maximum Intensity Projection (MIP), a Surface Shaded Display (SSD), or a Volume Rendering (VR).

In many sets of problems in the context of medical examinations and/or surgical interventions, only the shift and/or size change of the organ involved in one direction is significant (for example, the respiration shift of the liver because of the diaphragm movement along the body length axis or z-axis). As a result, for example, for a liver examination considered in the following, the ultrasound sensor is oriented in parallel to the axial layers pre-operatively obtained (CT-layers, MRT-layers, etc.) and—if a pure shift is present—driven up to the upper edge of the liver that is to be displayed well.

Equally as well, an anatomical marker in the form of a node, an aneurism, a sclerosis, a vascular bifurcation, etc. could also be driven into. If the organ—in this case the liver—has shifted relative to the preliminary examination, it appears on the monitor in the pre-operative (CT, MRT, etc.) image at another location. The pre-operative image and/or the organ section in the pre-operative image is then shifted virtually on the monitor so that the pre-operative image optimally matches at least in the organ region with the intra-operative image. At the push of a button and/or click of the mouse or by interaction on a touch screen, a recording is made of the z-difference between the current patient position in the intra-operative image and the patient position in the pre-operative image which is brought into the register matrix.

The modification of the register matrix causes a re-registration in the z-direction. This is sufficient if the organ has not changed with regard to its size. In order to control the size constant or if a changed length extension is known, the lower liver edge can also be displayed optionally with ultrasound as an axial layer. In turn, the possible organ shift in the pre-operative image is undone by a virtual shift on the monitor, until here as well, the z-positions of the lower edge match in the pre-operative and intra-operative image. This is acknowledged by the press of a button, mouse, etc., and brought into the matrix as scaling and translation by computer, together with the registration of the liver upper edge. In this way, in linear approximation, both the organ shift as well as the size change is taken into consideration, which could be confirmed by model tests.

In case of a periodic change, the re-registration is performed at certain time and location points (e.g., at stationary points or at certain points of a signal such as the EKG) and the tracking of the pre-operative image is dynamically performed in an analytical approximation (e.g. linearly interpolating), e.g., by the pneumatogram.

In summary, the present invention expands the conventional full registration in an advantageous way through a post-registration called re-registration, since the usual registration does not take into account a possible rotation, shift and compression and/or expansion of organs. The proposed re-registration causes a correlation of pre-operative and intra-operative images that is much more exact. The known basis-registration is important in spite of this since by it, a fundamental relatively exact positioning and orientation of both images can be created.

For the re-registration, a division is made among 4 modes:
1. Shift/scaling in x-direction
2. Shift/scaling in y-direction
3. Shift/scaling in z-direction
4. Rotation in three degrees of freedom.

In an operational sequence, a correction is best made only in one mode at a time. The process functions iteratively and can be applied repeatedly. In this way, either the equality of the fine registration is ensured or a renewed registration is performed that builds on the previous condition. Depending on the observed object relationship, a decision must be made in which direction (x, y, or z) shifts are present and whether at the same time a rotation is also to be considered. In most cases, all four influences can be reconstructed one after the other at least virtually so that a re-registration consisting of four steps produces a significantly improved matching of pre-operative and post-operative images. Usually, the correction in the x- and y-direction—e.g., to take into consideration the transverse expansion following a z-compression—is sufficient.

As opposed to a possible marker-based deformable re-registration with artificial markers, the process has the advantage that it is non-invasive and that adjustments can be made at points where insufficient access and/or because of the organ composition, no marker can be applied.

For a marker-based deformable registration with anatomical markers and/or an image-based deformable registration, the problem often exists that the organs under consideration have no such marks and/or structures or can not be seen in at least in one of the two displays. The above described embodiments of present invention solve this problem in that re-registration can simply be performed e.g., according to object limits. The intermediately positioned (linear) new scaling offers in practice sufficient accuracy.

With only one (mouse) click, a clearly improved repositioning of the pre-operative image can be achieved. Based on this, a combined repositioning and size adaptation of pre-operative and intra-operative images can, as an option, also be done. As opposed to the known process, the accuracy is increased in any case and in addition, a quality assurance is ensured to the extent that the process contains regular checks of the organ localization.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as C, C++, Java, assembler, or the like, with the various algorithms being implemented with any combination of data structures, objects, processes, routines or other programming elements. Furthermore, the present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

Reference Characters

1 object to be examined (patient)
2 special changeable object (organ)
3 patient's bed
4 (artificial or anatomical) marker
5 recording device for preliminary exam
6 recording device for main exam
7 monitor to display the organ of the preliminary exam in a pre-operative image
8 monitor to display the organ of the main exam in an intra-operative image
9 coupled display of the re-registered pre-operative and intra-operative image
10 re-registered pre-operative image
11 intra-operative image

1. A method for the coupled display of intra-operative and pre-operative images in medical imaging, comprising:
recording a pre-operative image of a region of a patient that is of interest;
recording an intra-operative image or an image sequence of the region of interest;
registering the pre-operative image with the intra-operative image;
coupling a display of the pre-operative image and the intra-operative image;
re-registering the pre-operative image, in case the pre-operative image and the intra-operative image do not match; and
repeating the steps of coupling the display and re-registering until an acceptable match of the pre-operative image and the intra-operative is present.

2. The method according to claim 1, wherein the registering is done in an image-based manner.

3. The method according to claim 1, wherein the registering is done in a marker-based manner, the method further comprising:
applying markers in the region of interest prior to recording the pre-operative image.

4. The method according to claim 1, further comprising:
utilizing a computer-based algorithm to determine when the acceptable match is present.

5. The method according to claim 1, wherein the step of the re-registration contains a 3D-rotation correction.

6. The method according to claim 1, wherein the step of the re-registration contains a shift correction.

7. The method according to claim 1, wherein the step of the re-registration contains a resealing correction.

8. The method according to claim 7, wherein the re-registration with resealing correction only relates to a region within the image and the regions outside remain are at least one of originally scaled and originally positioned.

9. The method according to claim 1, further comprising:
recording more than one intra-operative images during a periodic physiological movement cycle at defined points in at least one of time points and location points;
wherein
the re-registration of the pre-operative image and the coupled display relate to the more than one intra-operative images.

10. The method according to claim 1, further comprising:
obtaining a video sequence from the re-registered pre-operative images in that interpolation is done between the re-registered images on the basis of the physiological signals.

11. The method according to claim 10, wherein a cycle is defined as a corresponding physiological signal by an EKG, a blood pressure curve, or a pneumatogram.

12. The method according to claim 1, wherein the coupling of the display is done by adjacently displaying of both images arranged next to each other.

13. The method according to claim 1, wherein the coupling of the display is done by at least one of overlaying and cross-fading both the pre-operative image and the intra-operative image.

14. The method according to claim 1, wherein the pre-operative image is a 3D-image.

15. The method according to claim 1, wherein the intra-operative image is a 3D-image.

16. The method according to claim 1, further comprising:
utilizing, for recording the pre-operative image, magnetic resonance tomography, computer tomography, ultrasound, positron emission tomography, or nuclear medicine processes.

17. The method according to claim 1, further comprising:
utilizing, for recording the intra-operative image, an endoscope, laparoscope, ultrasound device, C-arc device, magnetic resonance tomography, computer tomography, positron emission tomography, or nuclear medicine processes.

18. An apparatus configured to couple a display of intra-operative and pre-operative medical imaging images, comprising:
a pre-operative recording device configured to record a pre-operative image of a region of a patient that is of interest;
an intra-operative recording device configured to record an intra-operative image of the region of the patient;
a display configured to present the pre-operative image and the intra-operative image to a user;
a mechanism configured to couple the display of the pre-operative image and the intra-operative image; and
an input device configured to allow the user to re-register the pre-operative image in case the pre-operative image and the intra-operative image do not match.
